(19) United States
(12) Patent Application Publication
(10) Pub. No.: US 2025/0255756 A1
(43) Pub. Date: Aug. 14, 2025

(54) Resizable Diaper

(71) Applicant: St Future International Ltd (Hong Kong), Hong Kong (CN)

(72) Inventor: YUMING LIANG, Hong Kong (CN)

(21) Appl. No.: 18/614,691

(22) Filed: Mar. 24, 2024

(30) Foreign Application Priority Data
Feb. 8, 2024 (CN) 202420290158.6

(51) Int. Cl. A61F 13/49; A61F 13/42; A61F 13/494

(52) U.S. Cl. CPC A61F 13/49007; A61F 13/42; A61F 13/494; A61F 2013/422

(57) ABSTRACT

A re-sizable diaper includes a three-dimensional absorption core, two anti-leakage portions respectively provided on the left and right sides of the three-dimensional absorption core, two longitudinal ends of the absorption portion respectively extending in a transverse direction to form a front waist portion and a rear waist portion, and elastic threads being seamed on the front waist portion and the rear waist portion; two side edges of the front waist portion are respectively bonded to two side edges of the rear waist portion; the two side edges of the rear waist portion are respectively provided with an extension portion, and a side edge of the extension portion are provided with an adhesive flap; the adhesive flap is adhered to the extension portion and can be torn off and adhered to the front waistline.

### TECHNICAL FIELD

The present disclosure relates to the technical field of diaper, more particularly to a re-sizable diaper.

### BACKGROUND

Diapers generally have a three-dimensional absorption core surrounding a crotch portion of the wearer. A left side and a right side of the three-dimensional absorption core are respectively provided with a leak-proof side edge. Two longitudinal ends of the absorption core extend transversely to fit the wearer's front waistline and rear waist waistline. In use, diapers are typically worn on the hip of the wearer, like underpants, to absorb excrement of the wearer, such as urine and menstrual blood. A typical three-dimensional absorption core is composed of three layers, a leakage layer in contact with the wearer's skin that allows excrement to pass through, an absorbent leakage prevention layer which absorbs excrement and prevents it from leaking, and a external air-permeable layer that is breathable.

Current diapers generally come in six sizes, including and not limited to NB, S, M, L, XL, and XXL, so as to be suitable for wearers of different ages and weights. Therefore, manufacturers must produce diapers in all common sizes, which increases costs. If the waistline of the diaper is smaller in size than the wearer, the leak-proof side will press against the waist and the crotch of the wearer, thereby leaving marks on skin of the wearer or even causing irritation. Yet if the waistline of the diaper is larger, the diaper cannot be well fitted with the hip parts of the wearer, and will leakage laterally. For cost saving and wearer-friendly purposes, it is necessarily to have a re-sizable diaper.

A disclosed patent of disposable hygiene product with a non-marking highly elastic protective belt, belongs to the technical field of sanitary articles, comprises of an elastic waistband, a front waist connector, and an absorbent part connecting the above two parts. Two sides of the absorption core are connected with shields that are parallel to each other. The shield consists of an abrasion-resistant outer layer and an elastic, waterproof membrane in the middle layer. The elastic waterproof membrane allows the elasticity of the shield to be evenly distributed, reducing the possibility of leaving marks on the wearer's skin by increasing the contact area between the shield and the user's skin.

However, the elastic waterproof membrane does not provide a fundamental solution to the defect of an inappropriate size diaper,

Hence, there is an urgent need to develop a re-sizable diaper so as to be suitable for wearers of different ages and weights.

### SUMMARY

In order to overcome the shortcomings of the prior art, the present disclosure provides a re-sizable diaper for wearers of different ages and weights, which can also streamline the categorization of diaper sizes in the manufacturing process, thereby saving cost.

The technical schemes of the present disclosure are as follows: a re-sizable diaper includes a three-dimensional absorption core surrounding the crotch portion of the wearer, two leak-proof portions respectively provided on the left and right sides of the three-dimensional absorption core, the two longitudinal ends of the absorption portion are extended in the transverse direction to form a front and a rear waistline to be attached to the wearer's waist, the front and a rear waistline are sewn with a number of elastic threads to fold into pleats; two side edges of the front waistline are respectively bonded to two side edges of the rear waistline to form an entire waistline and two leg cuffs of the re-sizable diaper; the two side edges of the rear waistline are respectively provided with an extension portion, and the side edge of the extension portion are provided with an adhesive flap; the adhesive flap is adhered to the extension portion and can be torn off and adhered on the front waistline.

The front and rear waistline are pleated with sewn-in the elastic threads to provide some degree of stretch during use, thereby the waistline takes an appropriate size to the wearer's waist.

If the waistline of the wearer is smaller than that of the diaper, there are two sub-cases: when the leg cuff of the diaper fits the leg circumference of the user, and the waistline of the diaper needs to be smaller to prevent leakage, both adhesive flaps are torn off from the extension portions and taped horizontally on the front waistline; and when the size of the leg cuff is bigger than the leg circumference of the user, the adhesive flaps are torn off from the extension portions and taped diagonally downwards on the three-dimensional absorption core.

If the waistline of the wearer is larger than that of the diaper, the joins of the two side edges of the front waistline and the two side edges of the rear waistline are torn open, the extension portion is used to increase the size of the entire waistline of the diaper and the adhesive flaps are adhere on the front waistline.

The two side edges of the front waistline and the two side of the rear waistline are respectively connected by sutures or adhesive glue to form a side-seam, which is secure for wearing as briefs and easy tear when needed.

The elastic threads are uniformly distributed in a transverse direction in the center of the front waistline to form a sparse elastic corrugation region, and uniformly distributed in a transverse direction on both sides of the front waistline to form a dense elastic corrugation region, thereby the center of the front waistline is relatively loose and the two sides are relatively tight.

The elastic threads pulled in tension to account for further re-sizing of the waistline when needed

The side of each of the leak-proof portions is sewn with the elastic threads to create a elastic corrugation region, the elastic corrugation region can be adjusted according to the crotch of the wearer.

The three-dimensional absorption core is provided with color-changing indicator. When excrement is absorbed, the indicator changes color, indicating to the caregiver whether the diaper needs to changed or not.

The tearing open of the side-seam between the front and rear waist enlarges the diaper, and the adhesive flaps on the extension part acts as belts to accommodate to the wearer. With the side-seam torn, the leg cuff opens creating a larger fit for the thighs.

The present disclosure possesses advantageous outcomes, including the ability to adjust both the waist and leg cuffs to accommodate wearers of diverse ages and weights, addressing issues such as skin irritation caused by smaller-sized diaper or leakage caused by larger-sized diaper, while simplifying the sizing of diaper.

### BRIEF DESCRIPTION OF THE DRAWINGS

FIG. 1 is a perspective view of the re-sizable diaper according to an embodiment of the present disclosure.

FIG. 2 is the other perspective view of the re-sizable diaper, showing that the adhesive flap 9 is adhesive on the front waist 3.

FIG. 3 is another perspective view of the re-sizable diaper, showing that the adhesive flap 9 is adhesive on the front waist 3.

FIG. 4 is another perspective view of the re-sizable diaper, showing that the two side edges 6 of the front waistline 3 are respectively torn off from the two side edges 7 of the rear waistline 4.

FIG. 5 is another perspective view of the re-sizable diaper, showing that the adhesive flap 9 is torn off from the extension portion 8 and the two side edges 6 of the front waistline 3 are respectively torn off from the two side edges 7 of the rear waistline 4.

FIG. 6 is another perspective view of the re-sizable diaper, showing that the adhesive flap 9 is used to connect the side edge 6 of the front waistline 3 and the side edges 7 of the rear waistline 4 by adhering on the front waistline 4, thereby enlarging the size of the waistline of the re-sizable diaper.

FIG. 7 is a front elevational view of the re-sizable diaper accordance with an embodiment of the present disclosure.

FIG. 8 is a rear elevational view of the re-sizable diaper accordance with an embodiment of the present disclosure.

### DETAILED DESCRIPTION

In order to demonstrate the purpose of the present disclosure, the technical scheme, and the technical effect of the re-sizable diaper fully, the present disclosure is further described below in connection with the embodiment. It should be known that the embodiment described herein are only used to interpretation of the present disclosure and are not intended to limit the present disclosure.

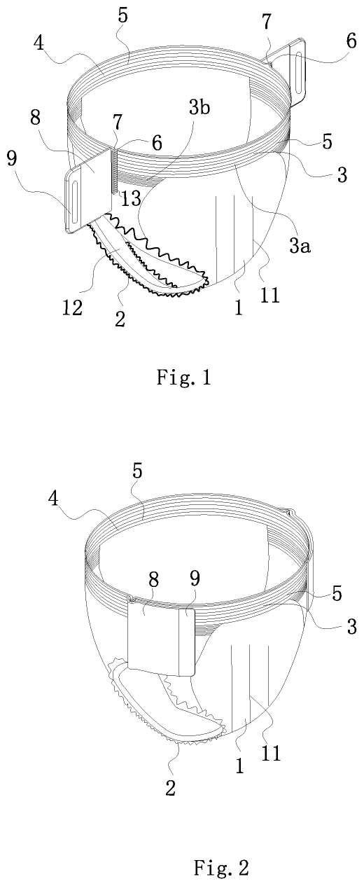

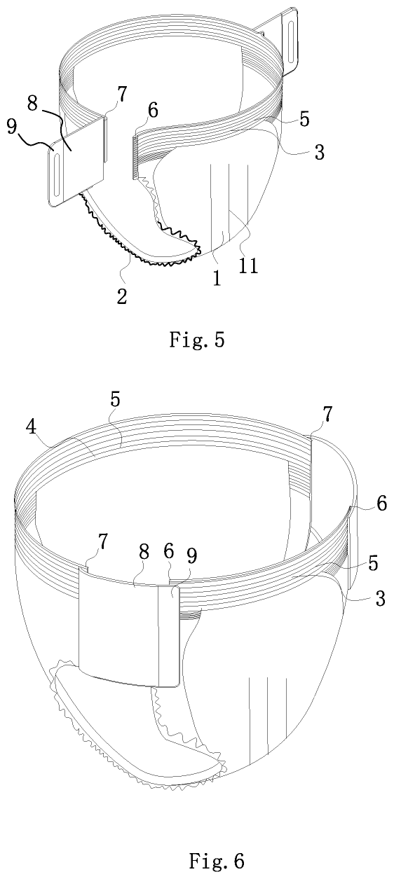

Referring to FIGS. 1-6, a re-sizable diaper includes a three-dimensional absorption core 1 surrounding a crotch portion of a wearer, two leak-proof portions 2 respectively provided on the left and right sides of the three-dimensional absorption core 1, two longitudinal ends of the three-dimensional absorption core 1 respectively extending in a transverse direction to form a front waistline 3 and a rear waistline 4 attached to a waist of a wearer, and a number of elastic threads 5 sewn on the front waistline 3 and the rear waistline 4 to fold them into pleats. Two side edges 6 of the front waistline 3 are respectively bonded to two side edges 7 of the rear waistline 4 to form an entire waistline and two leg cuffs 12 of the re-sizable diaper; the two side edges 7 of the rear waistline 4 are respectively provided with an extension portion 8, and the side edge of the extension portion 8 are provided with an adhesive flap 9; the adhesive flap 9 is adhered to the extension portion 8 and can be torn off and adhered on the front waistline 7.

The front and rear waistline 3 and 4 are pleated with sewn-in the elastic threads 5 to provide some degree of stretch during use, thereby the waistline of the re-sizable diaper takes an appropriate size to the wearer's waist.

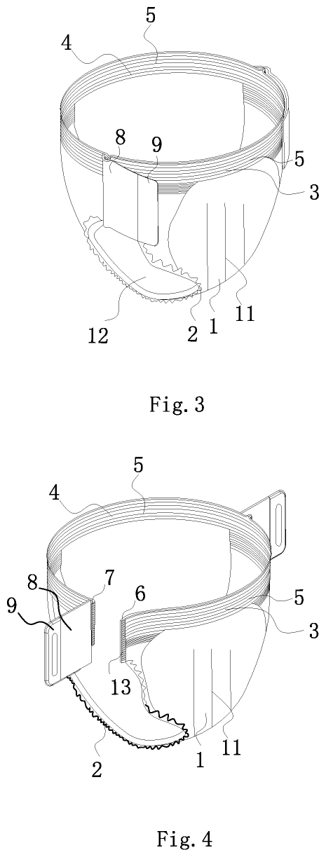

Referring to FIGS. 3 and 4, during use, the front waistline 3 and the rear waistline 4 automatically stretch to fit the waist of the wearer. If the front waistline 3 and the rear waistline 4 are too large for the wearer, the waistline of the re-sizable diaper can be narrowed by tearing the adhesive flaps 9 from the extension portion 8 and affixing them to an appropriate position horizontally or diagonally down on the front waistline 3. Referring to FIGS. 4, 5 and 6, If the sizes of the front waistline 3 and the rear waistline 4 are too small for the wearer, two sides where the front waistline 3 and the rear waistline 4 are bonded together can be torn off, the entire waistline can be increased by using the extension portion 8 and the adhesive flaps 10. Specifically, the adhesive flaps 10 is torn off the extension portion 8 and attached to the front waistline 3 in an appropriate position.

In addition, the two side edges 6 of the front waistline 3, the two side edges 7 of the rear waistline 4, and the left and right side edges of the three-dimensional absorption core 1 are enclosed to form two leg cuffs 12 to enclose the crotch of the wearer. If the size of each of the leg cuffs 12 is inappropriate for the crotch of the wearer, the adhesive flaps 9 may also be torn off from the extension portion 8 and attached to the front waistline 3 at an appropriate position. If the size of the leg cuffs 12 is too large for the crotch of the wearer, the joints between the two side edges 6 and 7 of the front waistline 3 and the rear waistline 4 are folded, and the adhesive flaps 10 is adhered to an appropriate position of the front waistline 3, thereby reducing the size of the leg cuffs 12. If the size of the leg cuffs 12 is too small relative to the size of the crotch of the wearer, the joints of the two side edges 6 and 7 of the front waistline 3 and the rear waistline 4 are torn off, the size of the leg cuffs 12 of the re-sizable diaper is increased by using the extension portion 8, the adhesive flaps 10 is torn off from the extension portion 8 and is adhered to the front waistline 3 at an appropriate position.

After use, the re-sizable diaper can be folded, the extension portion 8 can be stretched to bind the re-sizable diaper, and the adhesive flaps 9 can affixed to secure the re-sizable diaper from leakage of the excrement, thereby facilitating collection and disposal by cleaning personnel.

Referring to FIG. 1, in another embodiment, the two side edges 6 of the front waistline and the two side edges 7 of the rear waistline 4 are respectively connected by the sutures 13 or the adhesive glue. If necessary, the wearer can tear the joints of the two side edges 6 with the two side edges 7 easily. The re-sizable diaper and the sutures 13 are both made of a flexible material to contact the skin gently. Sutures 13 are a special design sturdy and secure when the diaper is worn as underwear pull-up piece as a perfect fit, yet for easy tear open when needed.

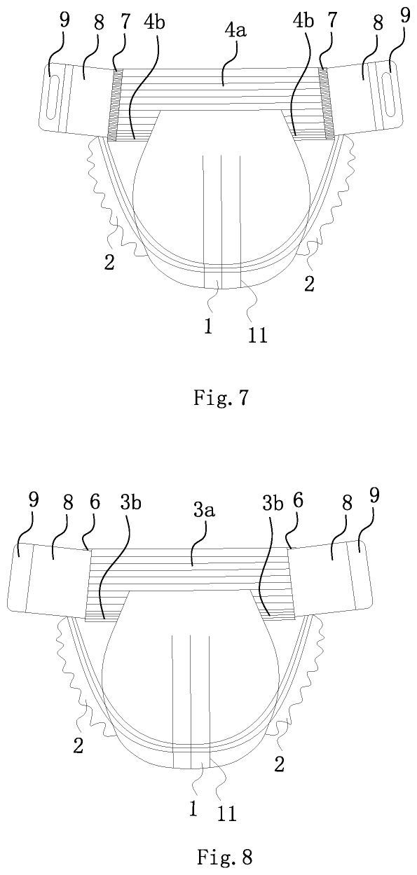

Referring to FIGS. 7 and 8, The elastic threads 5 are uniformly and transversely distributed in the center region of the front waistline 3 to form a sparse elastic corrugation region 3a and uniformly and transversely distributed on both sides of the front waistline 3 to form a dense elastic corrugation region 3b so that the center region of the front waistline 3 is relatively loose and the two side regions are relatively tight.

The elastic threads 5 are uniformly and transversely distributed in the center potion of the rear waistline 4 to form a sparse elastic corrugation region 4a and are uniformly and transversely distributed on both sides of the rear waistline 4 to form a dense elastic corrugation region 4b so that the rear waistline 4 is relatively tight.

The extension portion 8 is elastic in tension so that the waistline of the re-sizable diaper can be further adjusted.

The side edge of the leak-proof portion 2 is also seamed with the elastic threads 5, so that the leak-proof portion 2 also forms an elastic corrugation region, and the size of the elastic corrugation region can be adjusted according to the crotch of the wearer.

The three-dimensional absorption core 1 is provided with color-changing indicators 11, and after the three-dimensional absorption core 1 absorbs the excrement, the color-changing indicators 11 change color, so that a caregiver of the wearer can determine whether the wearer produces excrement according to the color.

It should be known that, although the aforementioned embodiment take a re-sizable diaper for generic wearers as an example, the re-sizable diaper in the present disclosure is not only applicable to wearers but also applicable to catamenial patient, adult nursing pants, and the like.

The above content is further elaborated on the disclosure combined with the preferred embodiment of the present disclosure, which cannot be identified that the embodiment of the present disclosure is only confined to these instructions. For general technical personnel belonging to the technical field of the present disclosure, without departing from the inventive concept, the architecture can be flexible and be derived from a series of products. Simple deduction or minor adjustments should be regarded as belonging to the protection scope that is determined by the submitted claims in the present disclosure.

1. A re-sizable diaper, comprising:
a three-dimensional absorption core;
two leak-proof portions, respectively provided on left and right sides of said three-dimensional absorption core;
a front waistline and a rear waistline: two longitudinal ends of said absorption portion respectively extending in a transverse direction to form a front waistline and a rear waistline; and,
a number of elastic threads, respectively seamed on said front waistline and said rear waistline to fold said front waistline and said rear waistline into fold pleats; two side edges of said front waistline are respectively connected to two side edges of said rear waistline;
wherein said two side edges of said rear waistline are respectively provided with an extension portion and a side edge of said extension portion is provided with an adhesive flap; said adhesive flap is adhered to said extension portion and can be torn off and adhered to said front waistline.

2. The re-sizable diaper according to claim 1, wherein said two side edges of said front waistline and said two side edges of said rear waistline are respectively bonded by sutures or adhesive glue.

3. The re-sizable diaper according to claim 1, wherein said elastic threads are uniformly and transversely distributed in a center region of said front waistline to form a sparse elastic corrugation region and are uniformly and transversely distributed in two side regions of said front waistline to form a dense elastic corrugation region so that said center region is relatively loose and said two side regions are relatively tight.

4. The re-sizable diaper according to claim 2, wherein said elastic threads are uniformly and transversely distributed in the center region of said rear waistline to form a sparse elastic corrugation region and are uniformly and transversely distributed in two side regions of said rear waistline to form a dense elastic corrugation region so that said center region is relatively loose and said two side regions are relatively tight.

5. The re-sizable diaper according to claim 1, wherein said extension portion is elastic in tension.

6. The re-sizable diaper according to claim 2, wherein said extension portion is elastic in tension.

7. The re-sizable diaper according to claim 1, wherein a side edge of each of said leak-proof portions is sewn with said elastic threads to form a elastic corrugation region.

8. The re-sizable diaper according to claim 2, wherein a side edge of said leak-proof portions is sewn with said elastic threads to form a elastic corrugation region.

9. The re-sizable diaper according to claim 1, wherein said three-dimensional absorption core is provided with color-changing indicators.

10. The re-sizable diaper according to claim 2, wherein said three-dimensional absorption core is provided with color-changing indicators.
